(19) United States
(12) Patent Application Publication
(10) Pub. No.: US 2017/0319534 A1
(43) Pub. Date: Nov. 9, 2017

(54) EUTECTIC ANESTHETIC TOPICAL COMPOSITIONS

(71) Applicant: Humco Holding Group, Inc., Texarkana, TX (US)

(72) Inventor: John Olin Trimble, San Antonio, TX (US)

(21) Appl. No.: 15/587,970

(22) Filed: May 5, 2017

Related U.S. Application Data
(60) Provisional application No. 62/332,576, filed on May 6, 2016.

(51) Int. Cl. A61K 31/245; A61K 47/24; A61K 47/18; A61K 47/14; A61K 47/10; A61K 9/06; A61K 31/167; A61K 31/085; A61K 31/08; A61K 9/107; A61K 47/34; A61K 47/12

(52) U.S. Cl. CPC A61K 31/245; A61K 47/34; A61K 47/24; A61K 47/18; A61K 47/14; A61K 47/12; A61K 9/06; A61K 31/167; A61K 31/085; A61K 31/08; A61K 9/107; A61K 47/10

(57) ABSTRACT

A eutectic anesthetic composition used to deliver pharmaceutical products topically as well as a method for producing the eutectic anesthetic composition, which may contain up to 80% additive ingredients. Preferred embodiments of the invention may include eutectic emulsion compositions which provide high viscosity/no separation due to API, are not temperature-sensitive, have no shear stress from the ointment mill/EMP, have no gumming up/stickiness or hardening, have improved active penetration and skin adhesion, and can use larger amounts of lipophilic active substances without lessening storage stability.

This application claims priority to U.S. Provisional Patent Application No. 62/332,576, filed May 6, 2016, entitled “Eutectic Anesthetic Topical Compositions,” the entire contents of which are hereby incorporated by reference.

### BACKGROUND

The present invention relates to a pharmaceutical composition comprising local anesthetics in base form and which is suitable for topical administration.

### Local Anesthetics

Local anesthetic agents are pharmacologically active agents that block nerve impulses conduction in sensory and motor nerve fibers when applied in therapeutically effective amounts. Their action is reversible, their use being followed by the complete recovery in nerve function with no evidence of structural damage to nerve fibers or cells.

Adjuvant anesthetics acting on voltage-gated channels play a fundamental role in the control of neuronal excitability. Alterations in the expression, distribution, and function of voltage-gated channels that occur following nerve injury or chronic inflammation have a profound effect on the firing of primary afferent neurons and contribute to the expression of pain behaviors. Examples of adjuvant anesthetics acting on voltage-gated channels include, but are not limited to, benzocaine, bupivacaine, dibucaine, diphenhydramine, etidocaine, gabapentin, lidocaine, mepivacaine, nifedipine, pregabalin, prilocaine, procaine, ropivacaine, tetracaine, and verapamil.

One approach to the prolongation of anesthesia involves the combination of several anesthetic agents. The most successful commercially available preparation for dermal anesthesia is the lidocaine-prilocaine cream named EMLA cream (U.S. Pat. No. 4,562,060). EMLA cream is an oil-in-water emulsion in which the organic phase is a eutectic mixture of lidocaine and prilocaine bases in water which is thickened with carbomer.

EMLA cream has solved the problem of obtaining a solution of a local anesthetic agent in the form of its base, where the concentration is higher than otherwise possible. Prilocaine in the form of its base and as such having a melting point of above 40 degrees C., is provided with lidocaine in the form of its base and as such having a melting point of below 40 degrees C., when brought and heated together form a homogenous oil.

Lidocaine and prilocaine salts are also formulated as a jelly, ointment, and spray for use as an anesthetic (U.S. Pat. No. 5,002,974). Unfortunately, these formulations are only effectively absorbed through mucosal surfaces, not the skin.

Topical anesthetics effective for relieving pain associated with skin conditions include hydrochloride salts of butamben, benzocaine, tetracaine, diperodon, dibucaine, lidocaine, diphenhydramine, methapyriline, tripelennamine, dimethisoquin, dyclonine, chloroprocaine, cocaine, mepivacaine, piperocaine, prilocaine, tetracaine, and pramoxine.

There have been several attempts trying to improve EMLA cream. One example is the topical anesthetic composition comprising a eutectic mixture of lidocaine and prilocaine in a lipophilic base (U.S. Pat. No. 5,993,836). It has been reported that this anesthetic formulation has significantly more rapid onset than similar transdermal anesthetics, such as EMLA cream.

### SUMMARY

The present invention relates to a topical pharmaceutical delivery composition, including matrices of a eutectic emulsion. The invention concerns at least one discontinuous phase in a continuous phase, with each discontinuous phase including a eutectic mixture of first and second pharmaceutically acceptable components which are both pharmacologically active agents and the continuous phase being provided by a pharmaceutically acceptable carrier. The discontinuous phases further comprise a polar oil and the continuous phase further comprises one or more lipophilic surfactants to obtain solubilizing characteristics. Such compositions exhibit a high concentration on skin, a deep anesthetic effect and a significantly more rapid onset of the anesthetic effect than comparable transdermal anesthetics.

By incorporating a specified concentration and ratio of a eutectic mixture comprising specified proportions of topical anesthetics such as lidocaine and prilocaine in a lipophilic base, a transdermal anesthetic formulation is produced which has significantly more rapid onset than comparable transdermal anesthetics, such as EMLA cream. The anesthetic works in as little as 10 to 40 minutes without occlusion.

Another example is the topical composition comprising an emulsion of at least one discontinuous phase in a continuous phase, or each discontinuous phase including a eutectic mixture of first and second pharmaceutically acceptable components which are both pharmacologically active agents and the continuous phase being provided by a pharmaceutically acceptable carrier, the eutectic mixture having a melting point below 40 degrees C. (U.S. Pat. No. 6,841,161). The topical composition may additionally comprise, in the eutectic mixture, a third or fourth pharmaceutically acceptable component.

Such topical compositions contain no co-solvent or additional oil phase, so that the eutectic mixture comprises each discontinuous phase of the emulsion.

Another example are compositions having a mixture of lidocaine, prilocaine and tetracaine, or their pharmaceutically acceptable salts. A preferred composition includes the following components in the indicated approximate w/w percentages: 1.5% lidocaine base; 1.5% prilocaine base; 4% tetracaine base and water. In some implementations, also included may be about 10% methylpyrrolidone; 2% dimethyl sulfoxide; 0.08% topical hyaluronidase; 1.5% guar gum; 1% polyoxyethylenesorbitan monolaurate; 0.5% polyoxyethylenesorbitan monooleate, and water to 100% (U.S. Pat. No. 8,609,722). Such compositions exhibits a high concentration on skin, a deep anesthetic effect and a significantly more rapid onset of the anesthetic effect than comparable transdermal anesthetics.

When tetracaine is as its hydrochloride, it can be dissolved in water and added to the previously prepared mixture of lidocaine and prilocaine. When tetracaine is as such, water is not necessary, so tetracaine can be added to the mixture of lidocaine and prilocaine directly. Optionally, the mixture of lidocaine, prilocaine and tetracaine can also be dissolved in alcohols.

A final example relates to a thermogelling pharmaceutical composition comprising local anesthetics in base form and which is suitable for topical administration (U.S. Pat. No. 9,254,263). The compositions further comprise a polyoxyethylene castor oil and one or more surfactants to obtain thermogelling characteristics.

Thermogelling has the meaning that the compositions are generally liquid with low viscosity at room temperature of at about 20 to 25 degrees C., but is a gel at body temperature at about 37 to 40 degrees C. The transition between liquid and gel does not necessarily need to be at body temperature, but preferably the composition shall undergo transition in the interval about 30 to about 37 degrees C. It is, however, important that the transition is sufficiently distinct at a defined temperature or at a fairly narrow temperature interval.

### Polar Oils

The scientific literature does not address the droplet size of the internal polar oil phase of topically applied emulsions. On the few occasions that refer to topical cream or lotion dosage forms, the indicated droplet size is in the range of a few to tens of microns (U.S. Pat. No. 4,529,601). A eutectic mixture of lidocaine and tetracaine is believed to produce a good local anesthetic effect that may not be achieved otherwise.

Emulsion droplet size is primarily influenced by the effect of the rotor speed, and was found to be almost independent of flow rate, especially for higher polar oil viscosities, and produced droplets as small as 0.2-0.6 mm after a single pass. The inlet droplet size ranged from 20 to 60 mm and was found not to greatly affect the outlet droplet size. Similarly the dispersed phase volume fraction was found not to affect the droplet size significantly for the range studied of 1-50 weight %, due to the presence of excess surfactant. Thus it may be concluded that the droplet size produced by the homogenization is largely a function of rotor speed and there is little advantage of improving the emulsification efficiency of earlier stages in the process, such as dispersion in stirred vessels.

Use of polar oils may help to improve solubility (U.S. Pat. No. 7,781,429). These compounds dissolve between 1.5 and 2.0 times more local anesthetics in base form.

An oil that is non-polar in character is assigned a low Hydrophile-Lipophile Balance (HLB) number below 9.0, and one that is polar is assigned a high HLB number above 11.0. Those in the range of 9.0-11.0 are intermediate. Below is a list of polar oils and their respective required HLBs:
C12-15 Alkyl Benzoate (HLB=13.0)
Castor Oil (HLB=14.0)
Isopropyl Myristate (HLB=11.5)
Isopropyl Palmitate (HLB=11.5)

### Lipophilic Surfactants

In addition to the polar oily phase, the emulsions may include a lipophilic surfactant. Lipophilic surfactants of interest include any type of surfactant that can be used for pharmaceutical formulations, including but not limited to, refined phospholipids, nonionic surfactants, or mixtures thereof. Refined phospholipids may include phosphatidylinocytol, phosphatidyl ethanolamine, phosphatidylserine, and sphingomyeline with phosphatidylcholine as a main ingredient. Nonionic surfactants of interest include, but are not limited to, polyethylene glycol, polyoxyalkylene copolymer, and sorbitan fatty acid esters. The amount of surfactant in an emulsion composition may vary (U.S. Pat. No. 8,535,692). In some instances, the amount of surfactant in the emulsion composition ranges from 0.05 to 5% by weight.

From experience, it is expected that the functions of emulsifiers might well be classified by HLB, and this is true. Below are some interesting 0/W emulsifiers with a high HLB number above 11.0:
Ceteareth-20 (HLB=15.2)
Cetearyl Glucoside (HLB=11.0)
Ceteth-10 (HLB=12.9)
Ceteth-20 (HLB=15.7)
Cocamide MEA (HLB=13.5)
Glyceryl Stearate (and) PEG-100 Stearate (HLB=11.0)
Isoceteth-20 (HLB=15.7)
Isosteareth-20 (HLB=15.0)
Lauramide DEA (HLB=15.0)
Laureth-23 (HLB=16.9)
Oleth-10 (HLB=12.4)
Oleth-10/Polyoxyl 10 Oleyl Ether NF (HLB=12.4)
Oleth-20 (HLB=15.3)
PEG-100 Stearate (HLB=18.8)
PEG-20 Methyl Glucose Sesquistearate (HLB=15.0)
PEG-60 Almond Glycerides (HLB=15.0)
PEG-8 Laurate (HLB=13.0)
PEG-80 Sorbitan Laurate (HLB=19.1)
Polysorbate 20 (HLB=16.7)
Polysorbate 60 (HLB=14.9)
Polysorbate 80 (HLB=15.0)
Polysorbate 85 (HLB=11.0)
Stearamide MEA (HLB=11.0)
Steareth-21 (HLB=15.5)

### Preparation of Eutectic Emulsion

Embodiments of a eutectic emulsion may be prepared, in one example, by mixing eutectic anesthetics with polar oil and lipophilic surfactant phases using a high-shear mixing method. Polar Oil Phase: in tank equipped with mixer, add Isopropyl Palmitate; turn on the mixer; add Lidocaine and Prilocaine and mix until dissolved. Lipophilic Surfactant Phase: in tank equipped with mixer, add Purified Water; turn on the mixer; heat to 75°-80° C.; add Ceteareth-20, Glyceryl Stearate (and) PEG-100 Stearate, and Polysorbate 60 and mix until dissolved; adjust pH to 9.0. Emulsion Phase: combine the Polar Oil Phase, and the Lipophilic Surfactant Phase in kettle with mixing; with mixing, add Caprylic/Capric Triglyceride, Lecithin 50% Solution, Hydroxyethyl Acrylate/Sodium Acryloyldimethyl Taurate Copolymer, Euxyl PE9010, and Dow Corning 200-350; mix for 1 hour; transfer to a storage vat.

Preferred embodiments of the current eutectic emulsion are composed of eutectic anesthetics, polar oils, lipophilic surfactants, penetrating agents, and thermogelling agents. These emulsions may be considered a third generation eutectic anesthetic (EMLA). The advantages of the present eutectic emulsion compared to the previous EMLAs are that it has readily-available chemical energy to form eutectic, rather than equipment-limited mechanical or thermal energy; it has improved particle size, improved active penetration, improved skin adhesion, and can use larger amounts of lipophilic active substances without lessening storage stability. It has sustained and controlled release of a wide range of pharmaceutical actives.

Dispersion of a lipophilic drug in the polar oil phase is conducted by dissolving the drug with a polar oil assigned a high HLB number above 11.0, to make Oil-in-Water (O/W) emulsions. Lipophilic drugs have an uptake capacity of about 30% to about 35%.

Dispersion of a hydrophilic drug in the lipophilic surfactant phase is conducted by dissolving the drug with a lipophilic emulsifier assigned a high HLB number above 11.0, to make Oil-in-Water (O/W) emulsions. Hydrophilic drugs have an uptake capacity of about 30% to about 35%.

### Characteristics of Eutectic Emulsion

Embodiments of the present eutectic emulsion should preferably emulsify eutectic mixtures of anesthetics with lipophilic surfactants in water. In this system, the local anesthetics are considered to be water-dissolved, surfactant-solubilized, and oil-dissolved in three separate phases. The dispersity of the polar oil phase was investigated by light microscopy and light-scatter spectroscopy. The majority of drops in the anesthetic emulsions were less than 1 pm in size. The concentration of freely dissolved drug in the aqueous phase of the emulsions was equal to the aqueous solubility of eutectic mixtures of anesthetics in a 1:1 ratio. At constant lipophilic surfactant ratio, increasing the total drug concentration in the emulsion resulted in an increase of the oil-dissolved fraction of local anesthetic, whereas the surfactant-solubilized fraction remained constant (Nyqvist-Mayer, A., Phase Distribution Studies on an Oil-Water Emulsion Based on a Eutectic Mixture of Lidocaine and Prilocaine as the Dispersed Phase, from “Journal of Pharmaceutical Sciences”, 1985).

Embodiments of the eutectic emulsion should preferably have long acting and topically effective local anesthetic agents. The non-ionized form of anesthetics is liquid in polar oils with low water solubility. A submicron o/w emulsion with Newtonian flow property was prepared with anesthetics as the oil phase. The kinetic stability of this emulsion was increased to prevent Ostwald ripening by addition of small amounts of hydrophilic emulsifiers to the water phase. The emulsion allowed a high in vitro release and permeation rate of anesthetics as well as a sufficient in vivo efficacy. To achieve a plastic property, thermogelling agents were added to the o/w emulsion resulting in significant control of the release and permeation rate of anesthetics (Welin-Berger, K., Formulations, Release and Skin Penetration of Topical Anesthetics, from “Comprehensive Summaries of Uppsala Dissertations from the Faculty of Pharmacy”, 2001).

Embodiments of the eutectic emulsion should preferably use eutectic mixtures of local anesthetics. The chemical stability of anesthetics were examined in advance and were found to be stable for more than 3 months. The release rate of anesthetics from the formulated emulsions were examined using epidermal membranes. Present studies suggest the potential for clinical use in easy, low-cost formulations (Zasshi, Y., Local Anesthetic Cream Prepared from Lidocaine-Tetracaine Eutectic Mixture, from “Pharmaceutical Society of Japan”, 2008).

### Etectic Emulsion in the Delivery of Local Anesthetics

Select formulations have graininess, gumming up, stickiness, and short duration of the anesthetic effect: Benzocaine 5% Ointment, Lidocaine 5% Ointment. Applied topically, local anesthetics reach peak effect at different times when applied to mucous membranes. Benzocaine is the fastest, followed by Lidocaine.

Select formulations have graininess, gumming up, stickiness, and long onset of the anesthetic effect: Dibucaine 1% Ointment, Tetracaine 0.5% Ointment. Tetracaine's effects can last up to 2 hours after topical application, and Dibucaine has the longest duration of action at 3-4 hours.

Select formulations have graininess, gumming up, stickiness, and minimal penetration of the skin epidermis: Gabapentin 10% Cream, Verapamil 15% Cream. Hardening of the final preparation and particles on ointment mill seen with Verapamil and Gabapentin.

Select formulations have low viscosity/separation due to API, and minimal penetration of the skin epidermis: Bupivacaine 1% Cream, Diphenhydramine 2% Cream, Etidocaine 2% Cream, Mepivacaine 3% Cream, Nifedipine 2% Cream, Pregabalin 2% Cream, Procaine 2% Cream, Ropivacaine 1% Cream. High-salt drugs and acids are avoided. The free base vs. the salt is used if possible. Shear stress from the ointment mill/EMP decreases the viscosity of the preparation as shear force increases.

Select formulations are commercially unavailable: Prilocaine.

In particular, this disclosure relates to compositions which may comprise polar oil phase(s) containing eutectic anesthetics and polar oils assigned a high HLB number above 11.0, and a lipophilic surfactant phase comprising lipophilic emulsifiers assigned a high HLB number above 11.0, penetrating agents and thermogelling agents. These polar oils are based on the ability of these materials to function differently from non-polar oils. Polar oils such as Castor Oil (HLB=14.0) are better solvents for eutectic anesthetics than non-polar oils such as Liquid Paraffin (HLB=10.0).

The current disclosure relates to a eutectic emulsion composition which could be used to deliver pharmaceutical products topically. The invention further comprises a method for producing the eutectic emulsion composition, which may contain a maximum combination of 35% eutectic anesthetics, and maximum internal phase(s) of 80% polar oils. Increasing the total concentration of these eutectic anesthetics and polar oils in the emulsions without changing the eutectic anesthetic/lipophilic surfactant ratio causes the freely dissolved fraction of eutectic anesthetic to decrease while the lipophilic surfactant emulsified fraction increases.

Preferred embodiments may include eutectic anesthetic compositions which have high viscosity/no separation due to API, are not temperature-sensitive, have no shear stress from the ointment mill/EMP, have no gumming up/stickiness, and no hardening. These rheological improvements are dependent on increased oil polarity and increased surfactant concentration.

The tables below show expiration dates for various topical anesthetics, including 3-month/40° C. stability data. Full-term controlled room temperature stability data are used to support the tentative expiry dating.

BENZOCAINE

| | | | | EXPIRY |
|---|---|---|---|---|
| INITIAL | 30 DAYS | 60 DAYS | 90 DAYS | DATING |
| 19.8% W/W | 20.5% W/W | 20.4% W/W | 19.6% W/W | ~1.2 YEARS |
| (99.0%) | (102.5%) | (102.0%) | (98.0%) | |

LIDOCAINE

| | | | | EXPIRY |
|---|---|---|---|---|
| INITIAL | 30 DAYS | 60 DAYS | 90DAYS | DATING |
| 10.0% W/W | 10.3% W/W | 10.1% W/W | 10.4% W/W | ~2.0 YEARS |
| (100.0%) | (103.0%) | (101.0%) | (104.0%) | |

TETRACAINE

| | | | | EXPIRY |
|---|---|---|---|---|
| INITIAL | 30 DAYS | 60 DAYS | 90 DAYS | DATING |
| 3.8% W/W | 4.1% W/W | 4.0% W/W | 4.0% W/W | ~2.0 YEARS |
| (95.0%) | (102.5%) | (100.0%) | (100.0%) | |

Preferred embodiments of the present eutectic anesthetic compositions have improved active penetration, improved skin adhesion, and can use larger amounts of lipophilic active substances without lessening storage stability. These efficacy improvements are dependent on decreased droplet size, increased penetration agents and increased thermogelling agents.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the HPLC chromatogram of Lidocaine (10%)/Tetracaine (4%)/Benzocaine (20%).

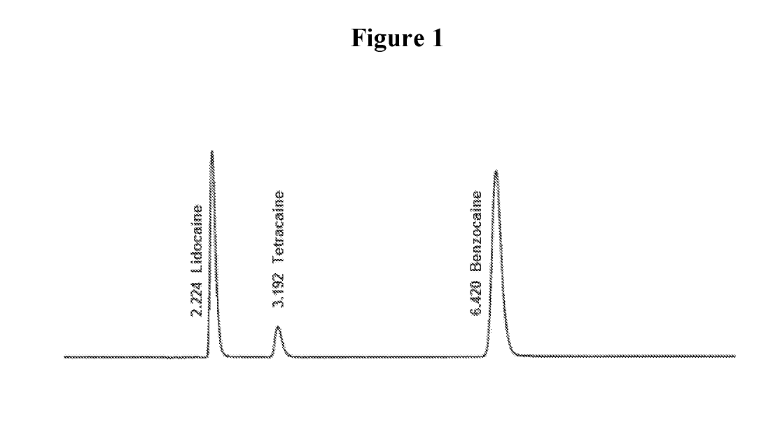

FIG. 2 shows the total percent Benzocaine that penetrated past the Stratum Corneum with an embodiment (“Eutectic Emulsion”) and a standard ointment (“Ointment”).

FIG. 3 shows the total percent of Lidocaine that penetrated past the Stratum Corneum with an embodiment (“Eutectic Emulsion”) and a standard ointment (“Ointment”).

FIG. 4 shows the total percent Tetracaine that penetrated past the Stratum Corneum with an embodiment (“Eutectic Emulsion”) and a standard ointment (“Ointment”).

### DETAILED DESCRIPTION OF PREFERRED EMBODIMENTS

One aspect of the current disclosure pertains to a eutectic emulsion composition which may be used to deliver anesthetics topically. The disclosure further comprises a method for producing the eutectic emulsion composition, which may contain up to 80% additive ingredients. Preferred embodiments may include eutectic emulsion compositions which provide high viscosity/no separation due to API, are not temperature-sensitive, have no shear stress from the ointment mill/EMP, have no gumming up/stickiness or hardening, have improved active penetration and skin adhesion, and can use larger amounts of lipophilic active substances without lessening storage stability.

### Composition

A preferred embodiment of the composition comprises at least one adjuvant anesthetic which preferably acts on voltage-gated channels. The adjuvant anesthetic may preferably include an aromatic ring, an intermediate chain, and an amine group, or a mixture thereof. Additional preferred embodiments may include eutectic mixtures of first and second pharmaceutically acceptable components which are both pharmacologically active. Examples of the adjuvant anesthetics include benzocaine, bupivacaine, dibucaine, diphenhydramine, etidocaine, gabapentin, lidocaine, mepivacaine, nifedipine, pregabalin, prilocaine, procaine, ropivacaine, tetracaine, verapamil, and mixtures thereof. The eutectic mixtures may be present in a concentration range of 0.1% to 35.0%, preferably 2.5% to 25.0%, and most preferably 5.0% to 15.0%.

A preferred embodiment further comprises polar oils assigned a high HLB number above about 11.0, preferably in the range of about 11.0 to about 20.0. The preferred polar oils may also have a droplet diameter of about 10 to about 100 microns. Preferred polar oils may also have viscosities ranging from about 5 to about 500 mPa s. Examples may include C12-15 alkyl benzoate, castor oil, isopropyl myristate, and isopropyl palmitate. The polar oils may be present in a concentration range of 5.0% to 80.0%, preferably 7.5% to 50.0%, most preferably 10.0% to 20.0%.

A preferred embodiment further comprises lipophilic emulsifiers assigned a high HLB number above about 11.0, preferably in the range of about 11.0 to about 20.0. Examples may include ceteareth-20, cetearyl glucoside, ceteth-10, ceteth-20, cocamide MEA, glyceryl stearate (and) PEG-100 stearate, isoceteth-20, isosteareth-20, lauramide, laureth-23, oleth-10, oleth-10/polyoxyl 10 oleyl ether NF, oleth-20, PEG-100 Stearate, PEG-20 methyl glucose sesquistearate, PEG-60 almond glycerides, PEG-8 laurate, PEG-80 sorbitan laurate, polysorbate 20, polysorbate 60, polysorbate 80, polysorbate 85, stearamide MEA, and steareth-21. The lipophilic emulsifier may be present in a concentration range of 1.0% to 20.0%, preferably 5.0% to 15.0%, most preferably 7.5% to 12.5%. In preferred embodiments, the lipophilic emulsifier may be caprylic/capric triglyceride, trimethylsiloxy-terminated dimethylsiloxane, C-20 Guerbet alcohol, glyceryl stearate, PEG 100 stearate, or mixtures thereof.

A preferred embodiment further comprises penetrating agents with phosphatidylcholine. Examples may include soy bean lecithin and egg yolk lecithin. The penetrating agent may be present in a concentration range of 0.1% to 5.0%, preferably 1.0% to 4.0%, most preferably 2.0% to 3.0%. In a preferred embodiment, penetrating agent may be lecithin 50% in isopropyl palmitate.

A preferred embodiment further comprises thermogelling agents with long-chain, straight or branched polymers. Examples may include acrylates/alkyl acrylate copolymer, acrylates/alkyl acrylate crosspolymer, acryloyldimethyltaurate copolymer and acryloyldimethyltaurate crosspolymer. The thermogelling agent may be present in a concentration range of 0.1% to 4.0%, preferably 1.0% to 3.0%, most preferably 1.5% to 2.5%. In a preferred embodiment, the thermogelling agent is a hydroxyethyl acrylate, sodium acryloyldimethyl taurate copolymer.

If desired, a saturated fatty alcohol such as myristyl alcohol, pentadecanol, cetyl alcohol, cetearyl alcohol, stearyl alcohol, nonadecanol, arachidyl alcohol, heneicosanol, behenyl alcohol, brassidyl alcohol, lignoceryl alcohol, ceryl alcohol and myricyl alcohol may be used in preferred embodiments. The fatty alcohol has the ability to provide a transitory effect on membrane permeability. The saturated fatty alcohol may be present in a concentration range of 0.1% to 5.0%, preferably 1.0% to 4.0%, most preferably 2.0% to 3.0%.

If desired, a moisturizer such as aloe vera oil, dimethicone, glycerin, phenyl trimethicone, vitamin E oil, and wheat germ oil may be used in preferred embodiments. The moisturizer stabilizes the skin prior to transmigration of the active agent and assists the skin to repair any damage. The moisturizer may be present in a concentration of 0.1% to 5.0%, preferably 1.0% to 4.0%, most preferably 2.0% to 3.0%.

If desired, an antimicrobial agent such as diazolidinyl urea, ethylhexylglycerin, methylparaben, phenoxyethanol, and propylparaben may be included in preferred embodiments. The antimicrobial agent is equally effective against bacteria, yeasts and mould fungi. The antimicrobial agent may be present in a concentration of 1.0% to 2.0%, preferably 1.2% to 1.8%, most preferably 1.4% to 1.6%. In a preferred embodiment, the antimicrobial agent is phenoxyethanol and ethylhexylglycerin.

Additional preferred embodiments may include one or more additional components, including but not limited to solvents or pH adjustment agents. In preferred embodiments, a solvent may be propylene glycol, and a pH adjustment agent may be triethanolamine.

### Methods

The Eutectic Anesthetic composition may be prepared by blending the proper amounts and ratios of all the required ingredients together.

One example of a method to prepare a preferred embodiment of the composition includes preparation as follows:
POLAR OIL PHASE: Charge a stainless steel tank with Lidocaine USP (anesthetic, component L from Table 1 below). Add Prilocaine USP (anesthetic, component M). Add Isopropyl Palmitate (polar oil, component N). Mix for 1 hour or until homogenous.
LIPOPHILIC SURFACTANT PHASE: Charge a stainless steel tank with lipophilic emulsifier component A (see Table 1 below). Add lipophilic emulsifier component B. Add lipophilic emulsifier component C. Add lipophilic emulsifier component D. Add Glyceryl Stearate & PEG 100 Stearate, lipophilic emulsifiers component E. Heat to 75-80° C.; mix for 20 minutes or until homogenous.
WATER PHASE: Charge a stainless steel tank with Purified Water, component F. Add Propylene Glycol, component G. Heat to 75-80° C.; mix for 20 minutes or until homogenous.
OIL PHASE: Charge a stainless steel tank with LIPOPHILIC SURFACTANT PHASE. Add POLAR OIL PHASE. Heat to 75-80° C.; mix for 20 minutes or until homogenous.
EMULSION PHASE: Charge the triple-motion kettle mixer/sweeper/emulsifier with WATER PHASE. Add OIL PHASE. Heat to 75-80° C.; turn on the mixer/sweeper/emulsifier to 60 Hz and mix, sweep and emulsify for 30 minutes or until homogenous. Cool to 25-30° C. Add Lecithin 50% in Isopropyl Palmitate (penetrating agent in polar oil, component H). Add antimicrobial agent component I. Add pH adjustment agent component J. Add thermogelling agent component K. Decrease the mixer/sweeper/emulsifier to 30 Hz and mix, sweep and emulsify for 30 minutes or until homogenous.

### EXAMPLE 1

A preferred embodiment of the present eutectic emulsion composition was prepared to contain the components in the table below, at the weight percentage amounts provided:

TABLE 1

| | Ingredient | Amount |
|---|---|---|
| A | LIPONATE ® GC (Vantage Specialty | 9.00 |
| | Ingredients, Warren, NJ), a caprylic/capric | |
| | triglyceride lipophilic emulsifier | |
| B | DOW CORNING ® 200 Fluid, 350 Cst. | 3.40 |
| | (Dow Corning Corporation, Auburn, MI), a | |
| | trimethylsiloxy-terminated dimethylsiloxane | |
| | lipophilic emulsifier | |
| C | EUTANOL ® G (BASF Corporation, | 3.40 |
| | Florham Park, NJ), a C-20 Guerbet alcohol | |
| | lipophilic emulsifier | |
| D | PROCOL CS20D (Protameen, Totowa, NJ), | 7.30 |
| | a ceteareth-20 lipophilic emulsifier | |
| E | Glyceryl Stearate & PEG 100 Stearate, | 7.30 |
| | lipophilic emulsifiers | |
| F | Purified Water | 45.57 |
| G | Propylene Glycol, a solvent | 4.60 |
| H | Lecithin 50% in Isopropyl Palmitate, a | 0.63 |
| | penetrating agent in a polar oil | |
| I | EUXYL ® PE9010 (Schulke & Mayr, | 1.00 |
| | Norderstedt, Germany), a phenoxyethanol | |
| | and ethylhexylglycerin antimicrobial | |
| J | TROLAMINE NF (Spectrum Chemicals, | 1.50 |
| | Gardena, CA), a triethanolamine pH | |
| | adjustment agent | |
| K | SIMULGEL NS (Seppic, Puteaux Cedex, | 1.10 |
| | France), a hydroxyethyl acrylate, sodium | |
| | acryloyldimethyl taurate copolymer | |
| | thermogelling agent | |
| L | Lidocaine USP | 2.50 |
| M | Prilocaine USP | 2.50 |
| N | Isopropyl Palmitate, a polar oil | 10.20 |

### EXAMPLE 2

Eutectic emulsion drug uptake capacity experiments were conducted by dissolving 20.0% Benzocaine, 10.0% Lidocaine and 4.0% Tetracaine into the embodiment set forth above (“Eutectic Emulsion”) in Example 1, in place of the 2.5% Lidocaine USP and 2.5% Prilocaine USP. The remaining components shown in Example 1 above were reduced accordingly, such as by 30.5%. Singular drugs are reported to have an uptake capacity of only 10% in Standard Ointment. Standard Ointment includes 95% white petrolatum and 5% white wax. The Eutectic Emulsion sample showed a steady simultaneous permeation of up to 100%, as shown in the table below. Permeation was measuring using Franz type diffusion cells and a normal human 3D model of epidermal tissue.

| Percutaneous Absorption | 12 | 24 | 36 | 48 |
|---|---|---|---|---|
| Testing | Hours | Hours | Hours | Hours |
| Benzocaine | 100% | 100% | 100% | 100% |
| Lidocaine | 8% | 16% | 23% | 23% |
| Tetracaine | 2% | 2% | 3% | 3% |

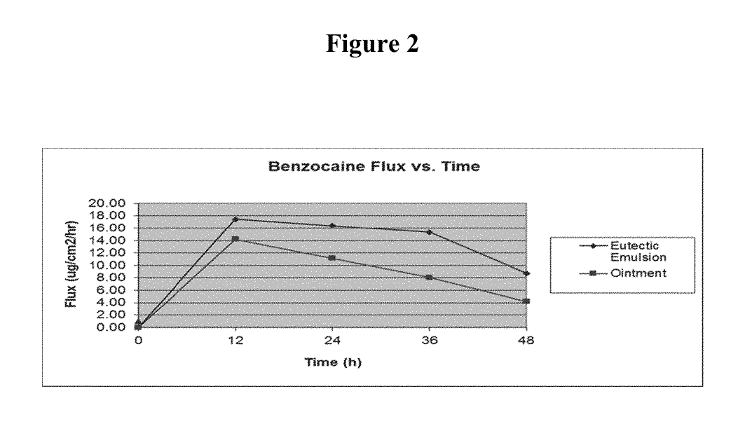

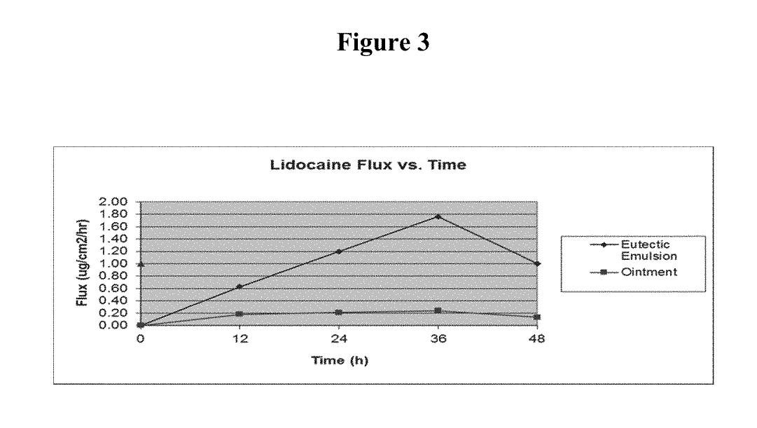

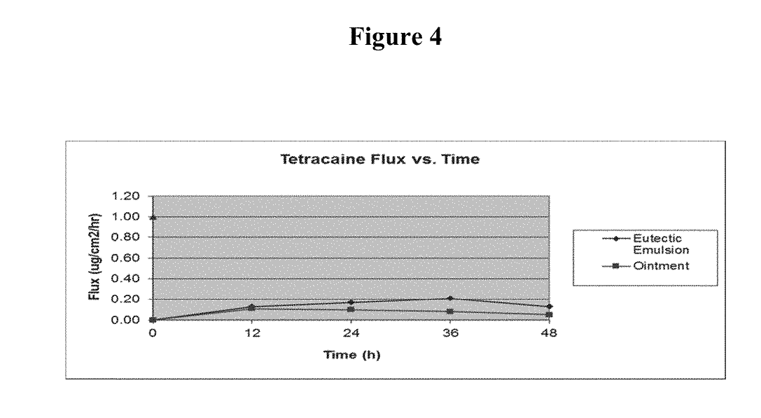

The percent of applied dose that penetrated past the stratum corneum with the Eutectic Emulsion was also up to 10.0 times more than a standard ointment containing the same anesthetic, as seen in FIGS. 2-4.

### REFERENCES CITED

The following references, to the extent that they provide exemplary procedural or other details supplementary to those set forth herein, are specifically incorporated herein by reference.

### U.S. Patent Documents

U.S. Pat. No. 4,529,601 to Broberg, et al., issued Jul. 16, 1985
U.S. Pat. No. 4,562,060 to Broberg, et al., issued Dec. 31, 1985
U.S. Pat. No. 5,002,974 to Geria, et al., issued Mar. 26, 1991
U.S. Pat. No. 5,993,836 to Castillo, et al., issued Nov. 30, 1999
U.S. Pat. No. 6,841,161 to Passmore, et al., issued Jan. 11, 2005
U.S. Pat. No. 7,781,429 to Schwarz, et al., issued Aug. 24, 2010
U.S. Pat. No. 8,609,722 to Fita, et al., issued Dec. 17, 2013
U.S. Pat. No. 9,254,263 to Sundberg, et al., issued Feb. 9, 2016

### REFERENCES

Nyqvist-Mayer, A., Phase Distribution Studies on an Oil-Water Emulsion Based on a Eutectic Mixture of Lidocaine and Prilocaine as the Dispersed Phase, from “Journal of Pharmaceutical Sciences”, 1985
Welin-Berger, K., Formulations, Release and Skin Penetration of Topical Anesthetics, from “Comprehensive Summaries of Uppsala Dissertations from the Faculty of Pharmacy”, 2001
Zasshi, Y., Local Anesthetic Cream Prepared from Lidocaine-Tetracaine Eutectic Mixture, from “Pharmaceutical Society of Japan”, 2008

What is claimed:

1. A eutectic anesthetic composition comprising:
at least one adjuvant anesthetic;
at least one polar oil having a droplet diameter in the range of about 10 microns to about 100 microns and having a Hydrophile-Lipophile Balance (HLB) number above about 11.0;
at least one lipophilic emulsifier having a HLB number above 11.0;
at least one penetrating agent comprising phosphatidylcholine; and
at least one thermogelling agent with long-chain, straight or branched polymers.

2. The eutectic anesthetic composition of claim 1, wherein the adjuvant anesthetic comprises an aromatic ring, an intermediate chain, and an amine group, or a mixture thereof.

3. The eutectic anesthetic composition of claim 1, wherein the adjuvant anesthetic is selected from the group consisting of benzocaine, bupivacaine, dibucaine, diphenhydramine, etidocaine, gabapentin, lidocaine, mepivacaine, nifedipine, pregabalin, prilocaine, procaine, ropivacaine, tetracaine, verapamil, and mixtures thereof.

4. The eutectic anesthetic composition of claim 1, wherein the at least one adjuvant anesthetic is a mixture of two adjuvant anesthetics selected from the group consisting of benzocaine, bupivacaine, dibucaine, diphenhydramine, etidocaine, gabapentin, lidocaine, mepivacaine, nifedipine, pregabalin, prilocaine, procaine, ropivacaine, tetracaine, and verapamil.

5. The eutectic anesthetic composition of claim 1, wherein the adjuvant anesthetic is present in a concentration range from about 0.1 to about 35.0 weight percent.

6. The eutectic anesthetic composition of claim 1, wherein the polar oil has a viscosity ranging from about 5 to about 500 mPa s.

7. The eutectic anesthetic composition of claim 1, wherein the polar oil is present in a concentration range from about 5.0 to about 80.0 weight percent.

8. The eutectic anesthetic composition of claim 1, wherein the polar oil has a HLB number ranging from about 11.0 to about 20.0.

9. The eutectic anesthetic composition of claim 1, wherein the lipophilic emulsifier has a HLB number ranging from about 11.0 to about 20.0.

10. The eutectic anesthetic composition of claim 1, wherein the lipophilic emulsifier is present in a concentration range from about 1.0 to about 20.0 weight percent.

11. The eutectic anesthetic composition of claim 1, wherein the penetrating agent is present in a concentration range from about 0.1 to about 5.0 weight percent.

12. The eutectic anesthetic composition of claim 1, wherein the thermogelling agent is present in a concentration range from about 0.1 to about 4.0 weight percent.

13. The eutectic anesthetic composition of claim 1, further comprising at least one saturated fatty alcohol.

14. The eutectic anesthetic composition of claim 13, wherein the saturated fatty alcohol is present in a concentration range from about 0.1 to about 5.0 weight percent.

15. The eutectic anesthetic composition of claim 1, further comprising at least one moisturizer.

16. The eutectic anesthetic composition of claim 15, wherein the moisturizer is present in a concentration range from about 0.1 to about 5.0 weight percent.

17. The eutectic anesthetic composition of claim 1, further comprising at least one antimicrobial agent.

18. The eutectic anesthetic composition of claim 17, wherein the antimicrobial agent is present in a concentration range from about 1.0 to about 2.0 weight percent.

19. The eutectic anesthetic composition of claim 1, further comprising at least one solvent.

20. The eutectic anesthetic composition of claim 1, further comprising at least one pH adjustment agent.

21. A eutectic anesthetic composition comprising:
at least one adjuvant anesthetic;
at least one polar oil having a droplet diameter in the range of about 10 microns to about 100 microns and having a Hydrophile-Lipophile Balance (HLB) number above about 11.0;
at least one lipophilic emulsifier having a HLB number above 11.0;
at least one penetrating agent comprising phosphatidylcholine;
at least one thermogelling agent with long-chain, straight or branched polymers;
at least one saturated fatty alcohol;
at least one antimicrobial agent;
at least one solvent; and
at least one pH adjustment agent.
